SEQUENCE LISTING (13 sequences)

SEQ ID NO: 1 - DNA, 25 bp, unknown
  primer
ctcgagtaatacgactcactatagg

SEQ ID NO: 2 - DNA, 46 bp, unknown
  primer designed from human p53 cDNA
ctcgagtaatacgactcactataggccgtccagggagcaggtagct

SEQ ID NO: 3 - DNA, 45 bp, unknown
  primer designed from human p53 cDNA
ctcgagtaatacgactcactataggccgggcgggggtgtggaatc

SEQ ID NO: 4 - DNA, 46 bp, unknown
  primer from human p53 cDNA
ctcgagtaatacgactcactatagggactgacattctccacttctt

SEQ ID NO: 5 - DNA, 49 bp, unknown
  primer from human p53 cDNA
ctcgagtaatacgactcactataggctctacctaaccagctgcccaact

SEQ ID NO: 6 - DNA, 49 bp, unknown
  primer designed from human p53 CDNA
ctcgagtaatacgactcactataggcctgttggctggtgggttggtagt

SEQ ID NO: 7 - DNA, 49 bp, unknown
  primer designed from human p53 cDNA
ctcgagtaatacgactcactataggaggaggggaagggtggggtgaaaa

SEQ ID NO: 8 - DNA, 20 bp, unknown
  primer
tgcgtgacattaaggagaag

SEQ ID NO: 9 - DNA, 18 bp, unknown
  primer
gctcgtagctcttctcca

SEQ ID NO: 10 - DNA, 24 bp, unknown
  primer
ccccctcctggcccctgtcatctt

SEQ ID NO: 11 - DNA, 22 bp, unknown
  primer
cgggcgggggtgtggaatcaac

SEQ ID NO: 12 - DNA, 336 bp, unknown
  relevant part of homo sapiens mitogen-activated kinase 1
cctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagca

SEQ ID NO: 13 - DNA, 276 bp, unknown
  relevant part of homo sapiens mitogen-activated kinase 3
ccactccgccaacgtgctccaccgagatctaaagccctccaacctgctcagcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagca

(19) United States
(12) Patent Application Publication
(10) Pub. No.: US 2008/0194028 A1
(43) Pub. Date: Aug. 14, 2008

(54) Highly Potent Hsirna Mixtures and Method for Gene Splicing

(75) Inventors: George Tzertzinis, Cambridge, MA (US); Celine Petit, Lyon (FR); George Feehery, West Newbury, MA (US)

Correspondence Address: HARRIET M. STRIMPEL, D. Phil., New England Biolabs, Inc., 240 COUNTY ROAD, IPSWICH, MA, 01938-2723, US

(73) Assignee: New England Biolabs, Inc., Ipswich, MA (US)

(21) Appl. No.: 10/589,144

(22) PCT Filed: Feb. 14, 2005
(86) PCT No.: PCT/US2005/004409

§ 371 Date: Jul. 12, 2007

Related U.S. Application Data
(60) Provisional application No. 60/543,880, filed on Feb. 12, 2004.
(60) Provisional application No. 60/551,558, filed on Mar. 9, 2004.

(51) Int. Cl. C12N 15/11; C07H 21/02

(52) U.S. Cl. 435/455; 536/23.1

(57) ABSTRACT

Compositions and methods for making the same and uses of the composition are provided where the composition has a plurality of dsRNA fragments with overlapping sequences, each fragment having a size in the range of 18-30 nt, such that the composition is formed by enzymatic digestion of one or more large dsRNAs and less than 2 nM of the composition is capable of specifically silencing expression of a target gene by at least 65% in transfected COS cells.

### BACKGROUND

RNA interference (RNAi) employing short double-stranded RNA (siRNA) is a powerful tool for silencing gene expression in mammalian cells (see for example, U.S. Pat. No. 6,506,559, International Publication No. WO 01/29058, International Publication No. WO 01/68836, International Publication No. WO 01/75164, U.S. Publication No. 2002-0114784, U.S. Publication No. 2003-0125281, U.S. Publication No. 2002-162126, U.S. Publication No. 20030108923, U.S. Publication No. 2002-0173478, Fire, et al. Nature 391:806-811 (1998); Yang, et al., Mol. Cell. Biol. 21:7807-7816 (2001), Elbashir, et al., Nature 411:494-498 (2001), Hammond et al. Nat. Rev. Genet 2:110-119 (2001), Sharp, Genes Dev. 15:485-490 (2001)).

A standard method for generating siRNA relies on an inherently expensive chemical synthesis of a pre-determined short sequence. Because not all parts of a target sequence are equally effective in silencing, it is necessary to generate multiple chemically synthesized fragments to identify those sequences which are effective (Holen et al. Nucleic Acids Res. 30:1757-1766, 2002)).

An alternative method for generating siRNA relies on in vitro transcription (see for example, Donze and Picard, Nucleic Acids Res. 30:1757-1766 (2002) and Paddison et al. Genes and Dev. 16:948-958 (2002)). While this approach does not require chemical synthesis, it remains necessary to choose and test individual short sequences to determine which are most effective. In previous studies, the standard siRNA concentration used was above 20 nM even as high as 200 nM (Kawasaki et al., Nucleic Acids Res 31:981-987 (2003); Elbashir et al., Nature 411:494-498 (2001); Wu et al. Cancer Res. 63:1515-1519 (2003)). If the concentration is too high, it can provoke non-specific responses (Semizarov et al. Proc. Natl. Acad. Sci. 100:6347-6352 (2003); Jackson et al. Nat. Biotechnol. 21:635-637 (2003); Persengiev et al. RNA 10:15-18 (2003)).

Several enzymatic approaches have been reported for cleaving double-stranded RNA (dsRNA) molecules into short fragments. An evolutionarily conserved enzyme, which is believed to cleave large dsRNA to produce siRNA in vivo, has been identified as Dicer (Bernstein, et al., Nature 409:363-366 (2001)). This enzyme contains a helicase motif, a PAZ (PIWI-ARGONAUT-ZWILLE) domain and a tandem repeat of a catalytic domain, which is RNaseIII-like. Drosophila extracts presumably containing Dicer mixed with large dsRNA in vitro produce short dsRNA in a range of sizes. The preferred size for RNAi applications in this mixture was determined by Tuschl et al. to be 21-23 nucleotides (International Publication No. WO 01/75164). Problems associated with using crude cell extracts containing a putative cleavage enzyme are, for example, that it is unclear what proteins in the mixture of proteins are necessary and sufficient to generate the observed effect. In addition, the extract is relatively inefficient at cleaving large dsRNA with only a relatively small amount of the starting material being cleaved to the desired size in vitro even under extended incubation times. (Paddison et al., Proc. Natl. Acad. Sci. 99:1443 (2002)).

More recently, mammalian Dicer has been obtained recombinantly from baculovirus cell expression systems. Lysates of recombinant Dicer produced in baculovirus infected insect cell cultures are reported to generate short dsRNA fragments from large dsRNA in the presence of a magnesium buffer. The purified siRNA fragments were used for “silencing” the expression of cognate genes in cultured mammalian cell lines (Myers et al. Nature Biotechnology, 21:324-328 (2003)). Limitations of this approach include the cost of baculovirus expression systems, the incomplete digestion of dsRNA starting material and the need for gel based or other purification step to eliminate precursor RNA prior to performing silencing experiments.

An alternative enzymatic approach for generating small dsRNAs has been to use E. coli RNaseIII in the presence of magnesium ions to partially digest large dsRNA (Yang et al. Proc. Nat'l. Acad. Sci. USA 99:9942-9947 (2002)). Problems associated with this approach include low recovery amounts of the double-stranded fragments in a specific size range larger than about 15 nt and the associated inconvenience of titration to avoid over or under-digestion. Unless digestion is carefully monitored, RNaseIII in the presence of magnesium ions cleaves large dsRNA into very small fragments that are generally considered to have no known use in RNAi. Careful titration and timing of the partial digest at best yielded a smear throughout a gel after which a particular size fraction could be recovered for use in RNA silencing in cultured mammalian cells (Yang, et al., Proc. Nat'l. Acad. Sci. USA 99:9942-9947 (2002)). A problem with this approach is the lack of certainty with respect to (a) an end product where the end product relates to yield of a dsRNA having a particular size larger than about 15 nucleotides and (b) the extent of representation of the large dsRNA sequence in the cleavage products. The latter may be important since not all parts of the sequence of a long dsRNA are thought to be equally effective in gene silencing and important sequences may be under-represented while unimportant sequences may be over-represented.

Although synthetic siRNAs and also plasmid generating siRNAs that can effectively suppress the expression of endogeneous gene have been reported (Elbashir et al. Nature 411:494-498 (2001)), the efficiency of such siRNAs is highly dependent on target position ((Holen et al. Nucleic Acids Res. 30:1757-1766 (2002)).

Because gene silencing has become a methodology of great importance in understanding molecular functions in cells and organisms, it is desirable to have a rapid, cost effective and reliable method for generating double-stranded RNA suitable for silencing of any gene. Furthermore, since the specificity of synthetic and hairpin-derived siRNAs has been recently questioned especially in the context of transfection at a concentration that is currently required to obtain effective silencing (Semizarov et al. Proc. Natl. Acad. Sci. 100:6347-6352 (2003); Jackson et al. Nat. Biotechnol. 21:635-637 (2003); Persengiev et al. RNA 10:15-18 (2003); Bridge et al. Nat. Genet. 34:263-264 (2003)), it is desirable to obtain and use siRNAs that are highly effective at low transfected concentrations to ensure specificity.

### SUMMARY

In an embodiment of the invention, a composition is provided that is characterized by a plurality of dsRNA fragments having overlapping sequences, each fragment having a size in the range of 18-30 nt, wherein the composition is formed by enzymatic digestion of one or more large dsRNAs and wherein less than 2 nM of the composition is capable of specifically silencing target gene expression by at least 65% in transfected COS cells or, for example, by at least 70% or, for example, by at least 80%.

In a further embodiment of the invention, the large dsRNA has a size of at least 100 nt in length. Additionally, the plurality of fragments may constitute at least 5 fragments, more particularly, more than 10 fragments.

In another embodiment, the sequence of the large dsRNA shares identity with a first portion of a mRNA sequence such that the plurality of dsRNA fragments derived therefrom have a greater gene silencing activity at less than 2 nM than a second plurality of fragments having sequence identity with a second portion of the mRNA.

In another embodiment, the plurality of dsRNA fragments have a greater gene silencing activity at less than 2 nM than any single fragment in the composition.

The composition described above which is a product of enzyme digestion is in certain embodiments a product of digestion with RNaseIII in a manganese buffer or a mutant RNaseIII. Double-stranded RNA fragments derived from digestion of a plurality of dsRNAs may have sequence identity with a non-contiguous or a contiguous region of the mRNA.

In another embodiment of the invention, a method is provided of preparing a composition as described above, where the method has the following steps: (a) transcribing at least one RNA molecule having a sequence identity with a portion of a target gene, to form a large dsRNA; (b) cleaving the large dsRNA into a mixture of overlapping fragments having a size in the range of 18-30 nt by means of RNaseIII or mutants thereof; and (c) determining whether less than 2 nM of the fragment mixture can silence at 65% of expression of the target gene in COS cells after transfection.

In an embodiment of the invention, a method of silencing gene expression is provided which includes the steps of (a) cleaving with an enzyme, a large dsRNA having sequence identity with a target gene, wherein the enzyme is RNaseIII or a mutant thereof and the cleavage product is a set of overlapping fragments of dsRNA in which greater than 80% of the fragments have a size of less than about 40 nt; (b) transfecting cells with the cleavage product of step (a) without size fractionating the product; and (c) obtaining at least 65% silencing of expression of the target gene.

In an additional embodiment, step (b) of the above method may include transfecting cells with less than 2 nM of the cleavage product of step (a). Additionally, the large dsRNA may be cleaved by RNaseIII in the presence of manganese buffer.

### LIST OF FIGURES

FIG. 1 shows a schematic representation of p53 full-length cDNA (Acc. Number NM_{—}000546) and 11 segments of sizes varying from 60-600 nt distributed over the entire sequence. The segments are identified by coordinates. Restriction enzyme (AciI) digestion of the p53 gene produced 8 segments (C2, C4,11, C6, C17, C10,12,19, C9, C20 and C14,18).

“C” corresponds to constructs that were obtained by cloning.

“P” corresponds to constructs produced by PCR amplification.

FIG. 2A shows a sequence of steps leading to transfection and silencing of gene expression.
(1) Insertion of a DNA fragment into a vector under the control of an inverted T7 promoter pair or amplification from cDNA.
(2) Amplification of DNA and its immobilization using streptavidin beads.
(3) In vitro transcription of immobilized DNA to produce large dsRNA.
(4) RNaseIII digestion of the large dsRNA in the presence of Mn²⁺ buffer to form an hsiRNA mixture.
(5) Transfection of cells with an hsiRNA mixture for gene silencing.

FIG. 2B shows an agarose gel with bands corresponding to C1 and C4 dsRNA.

FIG. 2C shows a polyacrylamide gel with bands corresponding to C1 and C4 hsiRNA mixtures for C1 and C4.

FIG. 3A shows a Western Blot of cell lysates obtained 48 hours after transfection with 10 nM of different hsiRNA mixtures. The target protein p53 and the non-target control beta-actin were detected with the corresponding antibodies where:

Lane 1 is an unrelated cyclic AMP response element binding protein (CREB) hsiRNA mixture.

Lane 2 is a control fluorescent synthetic siRNA (fluo).

Lane 3 is a C1 hsiRNA mixture (C1 has a length of 66 nt).

Lane 4 is a C20 hsiRNA mixture (C20 has a length of 327 nt).

Lane 5 is a C6 hsiRNA mixture (C6 has a length of 333 nt).

Lane 6 is a C14 hsiRNA mixture (C14 has a length of 313 nt).

FIG. 3B shows Western blots for p53 silencing with 25 nM and 10 nM concentrations of hsiRNA mixtures as indicated from P2, P3, P4 and C10.

FIG. 4 shows the standardization of quantitative real-time PCT (QPCR) for calculating the relative expression levels of p53 mRNA with specific primers.

QPCR amplification standard curves for p53 (target) and beta actin (control) are shown. The threshold cycle (Cₜ) obtained is plotted as function of the corresponding input total RNA in the reverse transcription.

FIG. 5A shows QPCR analysis of the knockdown of p53 mRNA levels in a titration of the hsiRNA mixture targeting segment C2 from 1.5 nM to 150 nM transfection concentrations.

FIG. 5B shows a Western Blot of the p53 protein in the titration experiment of FIG. 5A.

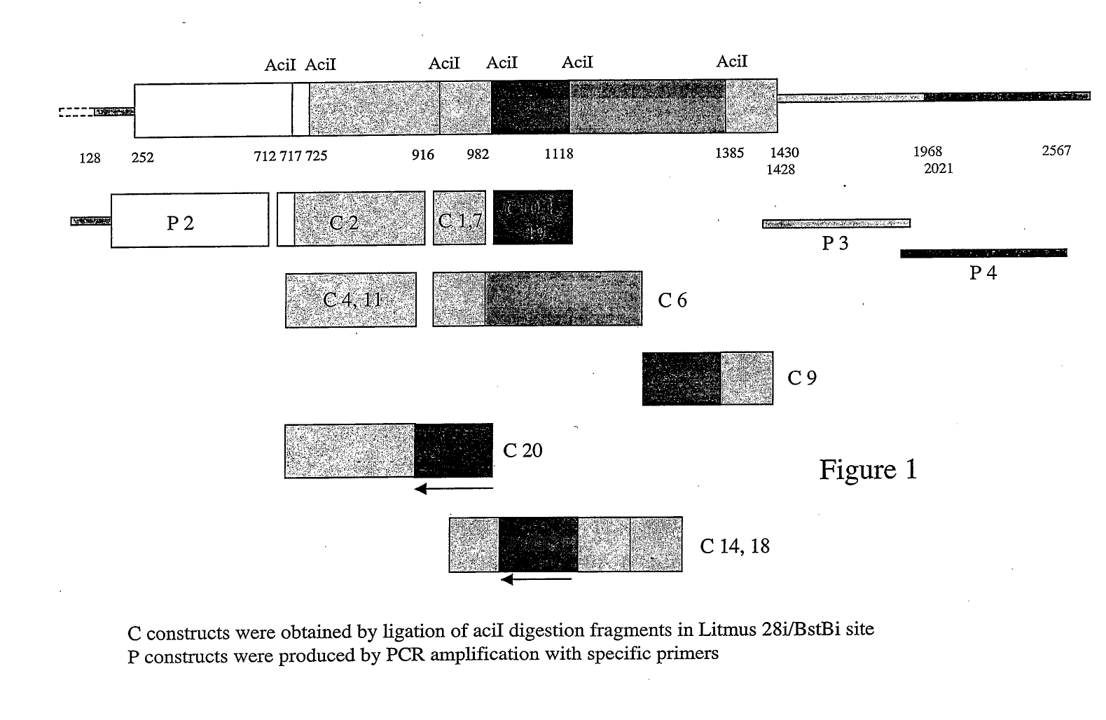

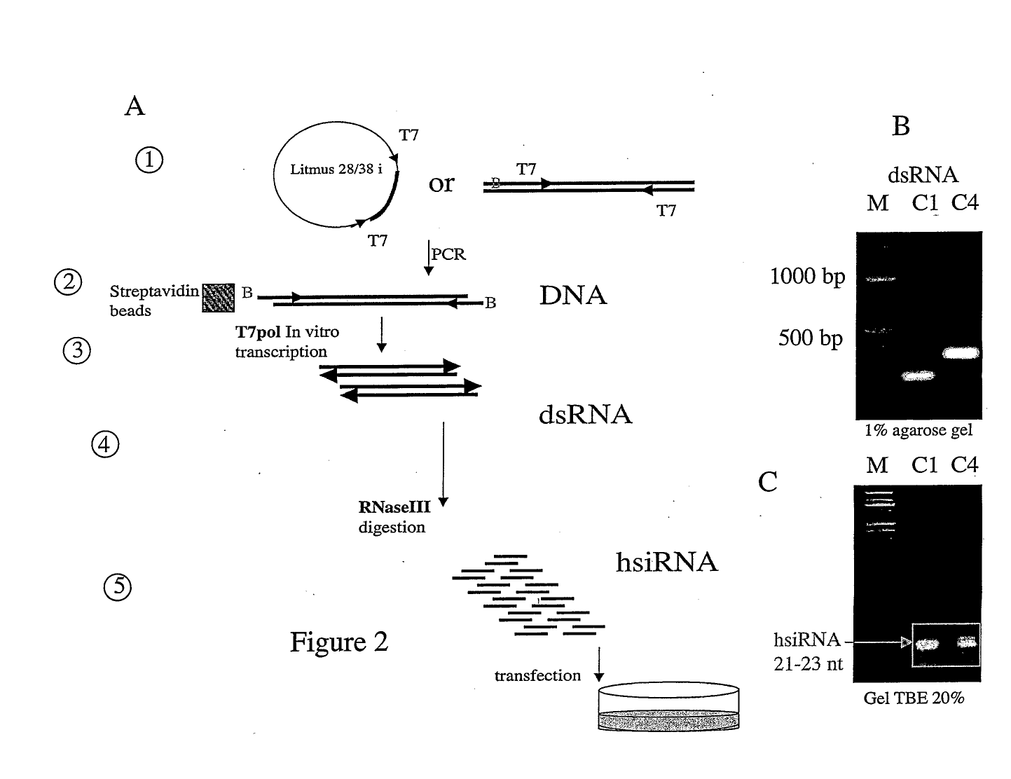

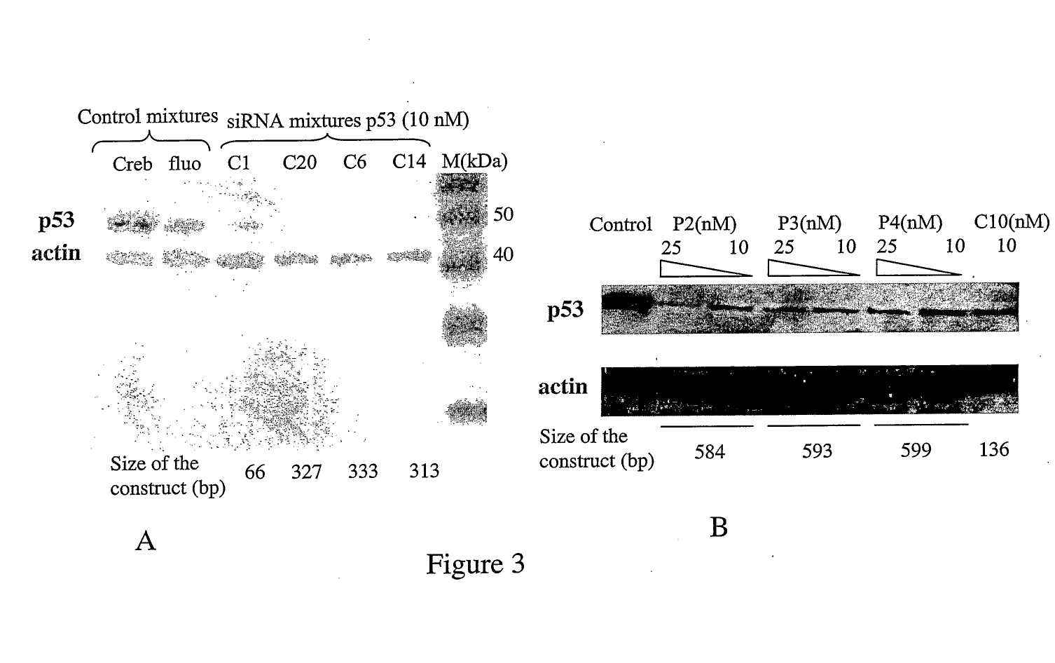

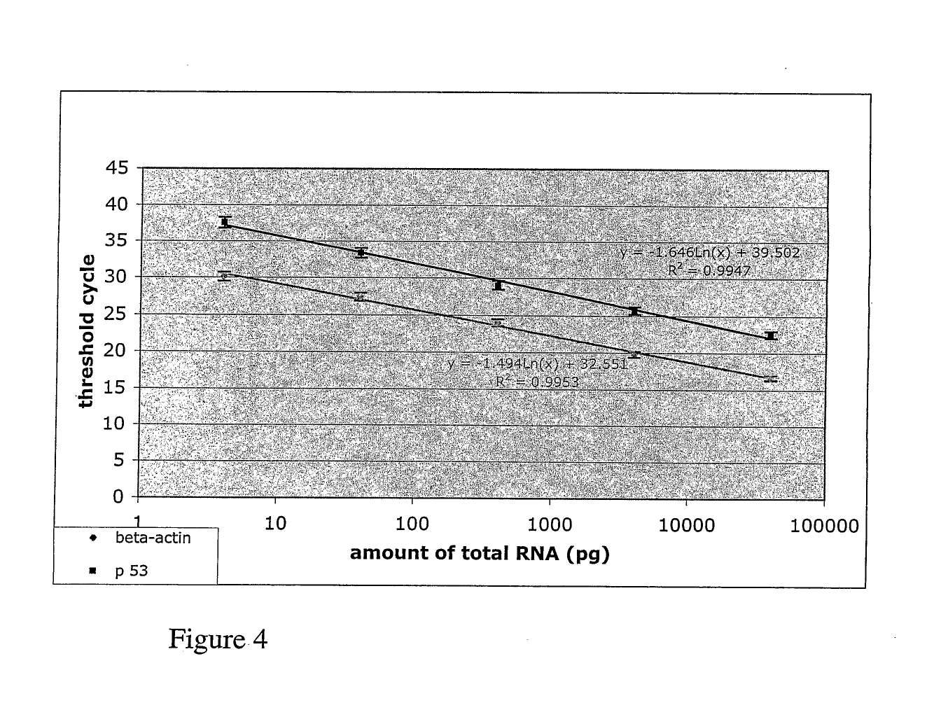

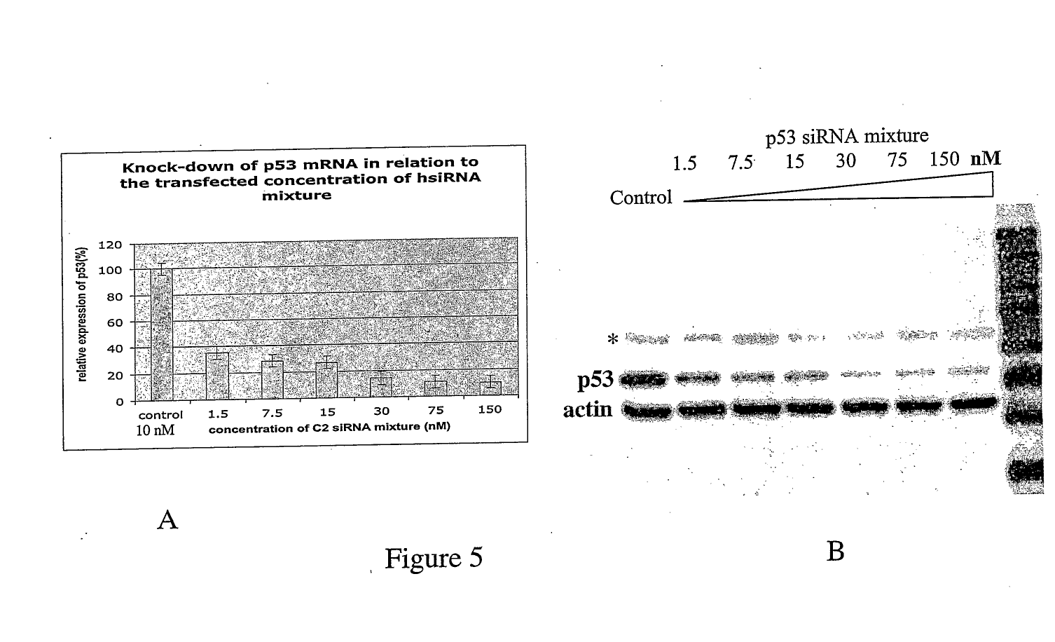

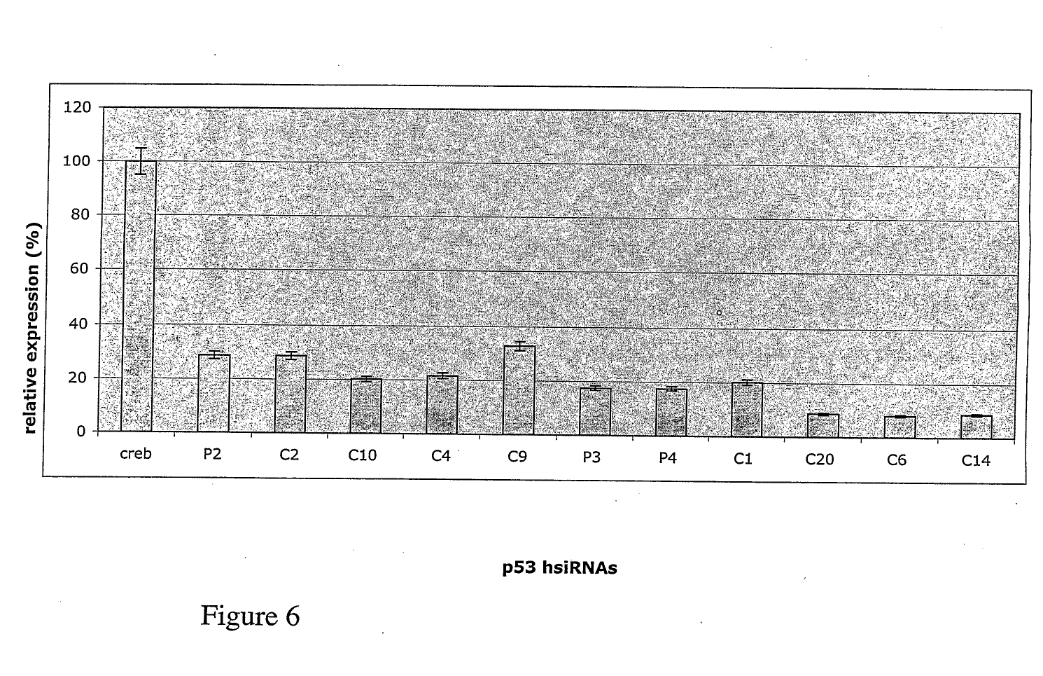

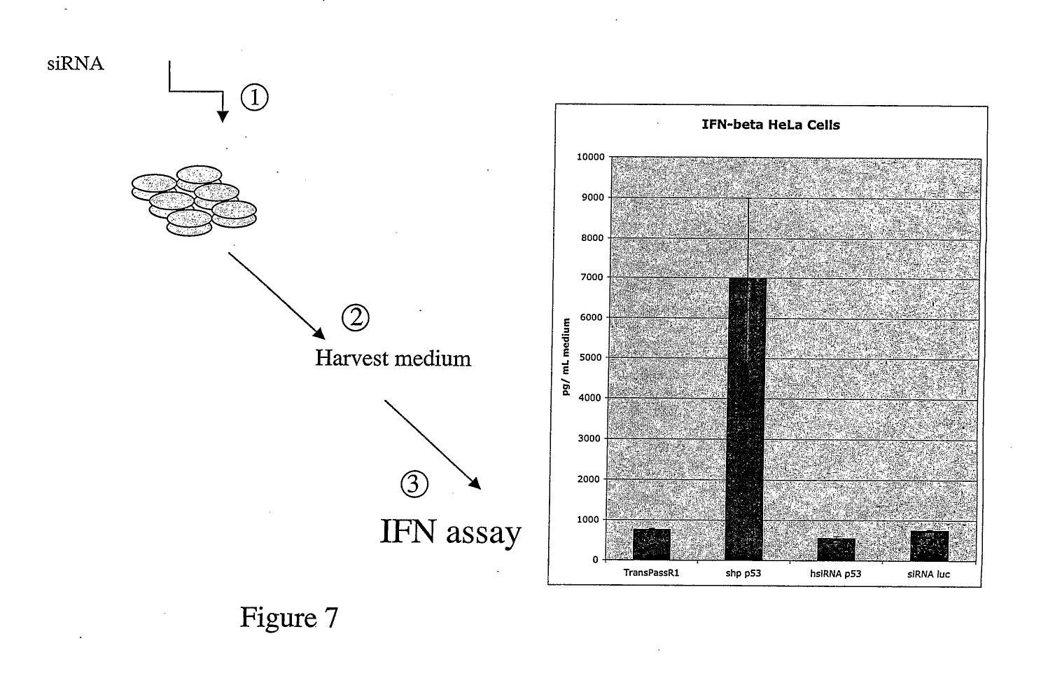

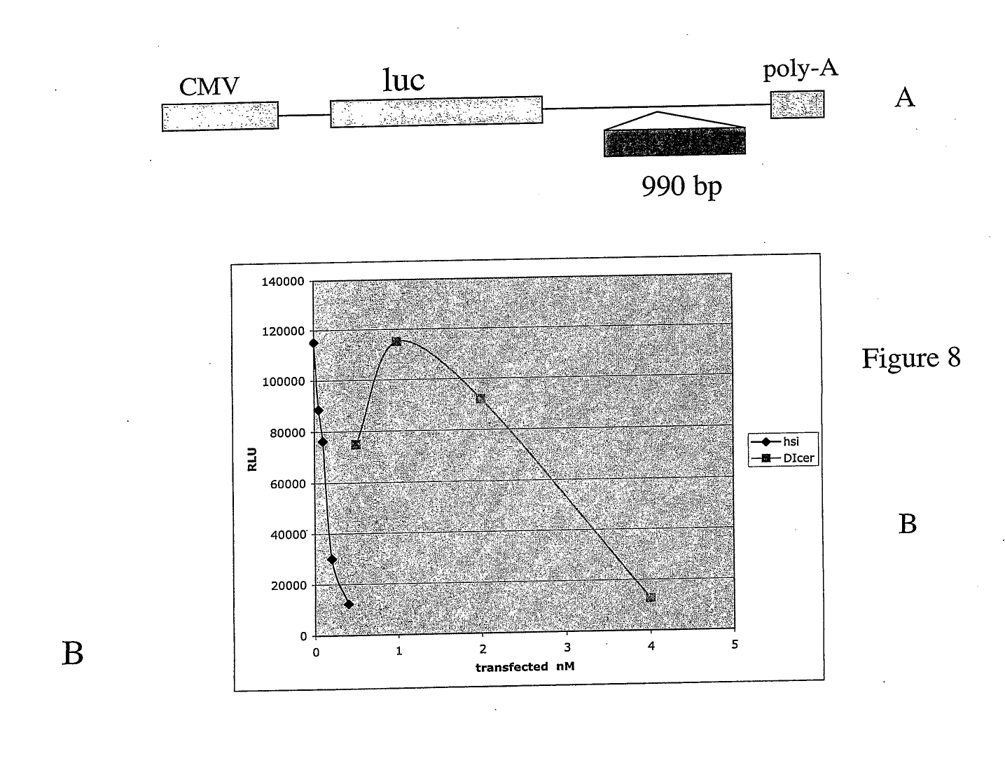

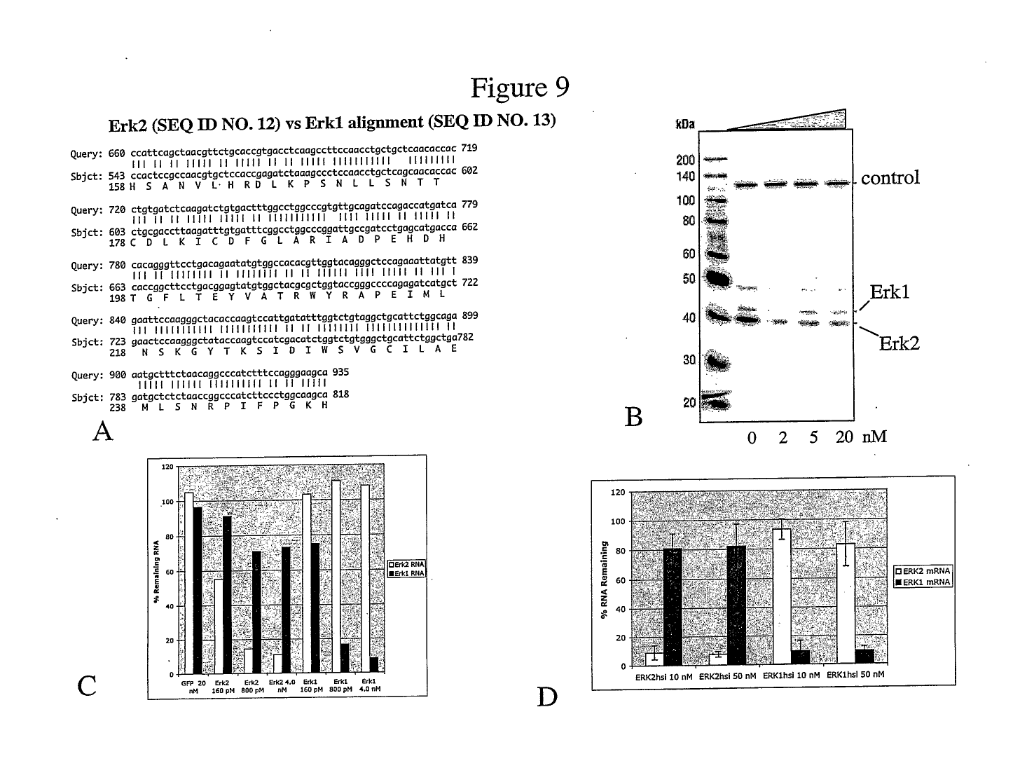

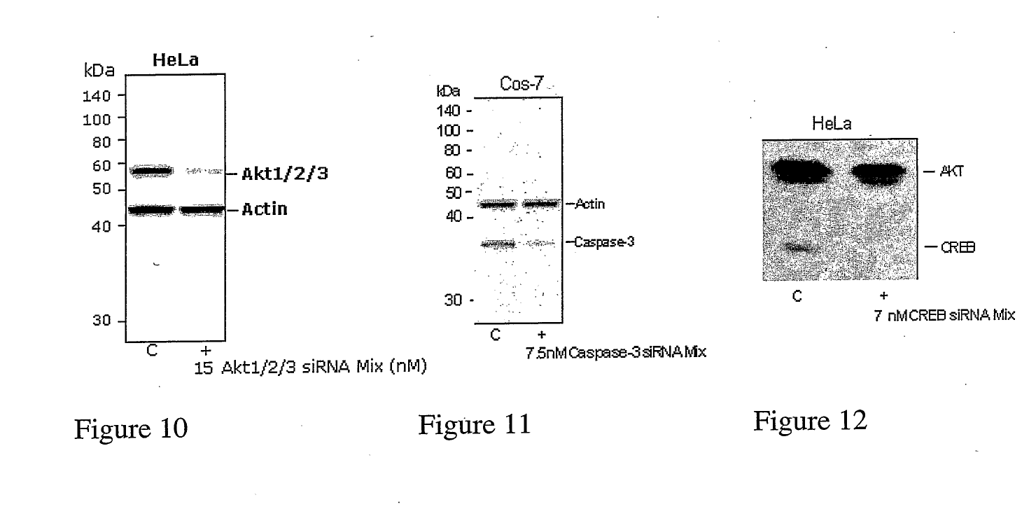

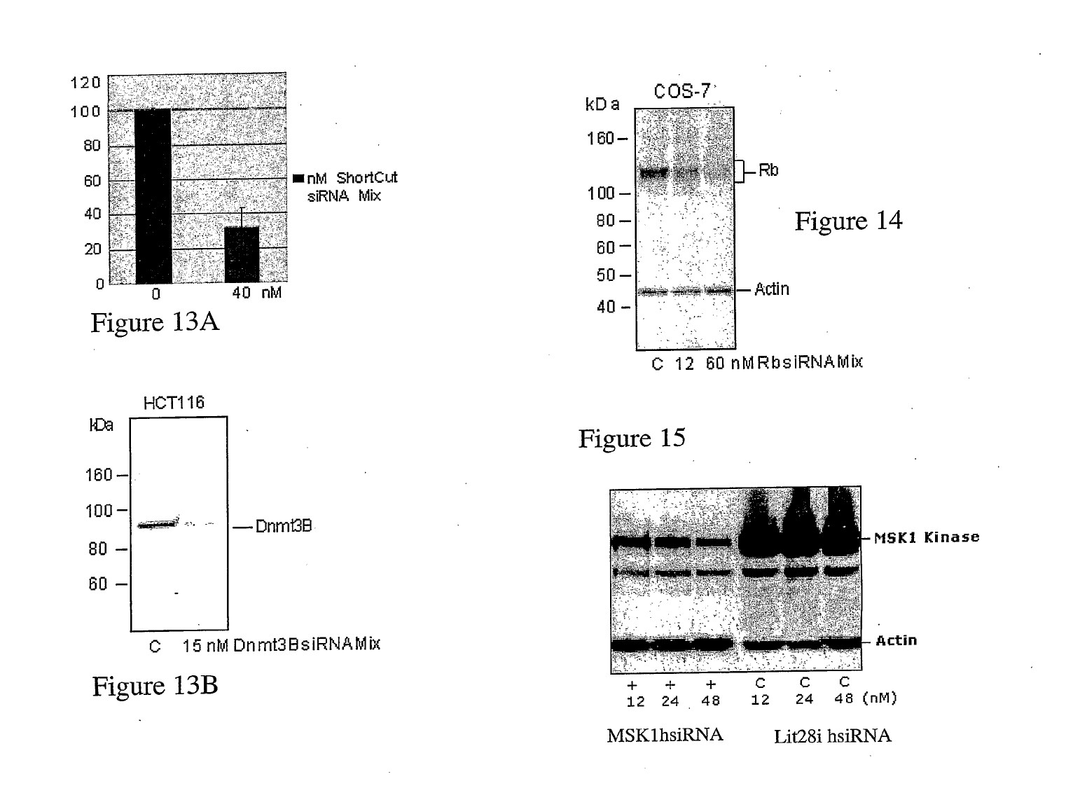

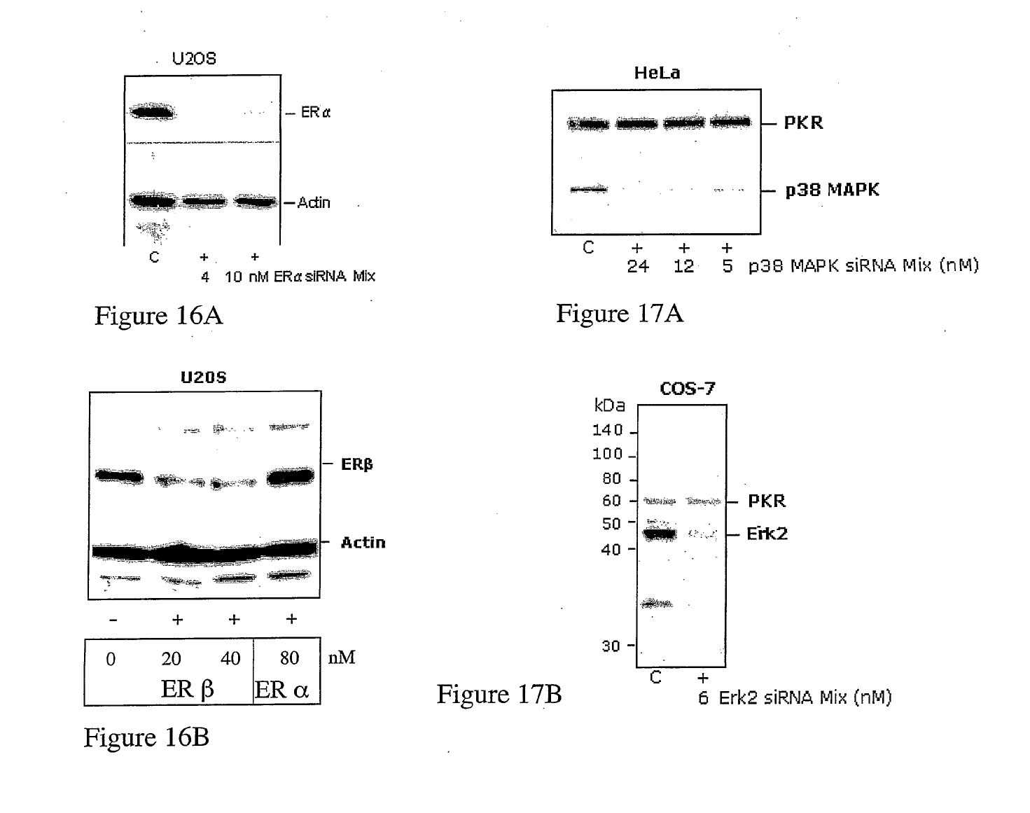

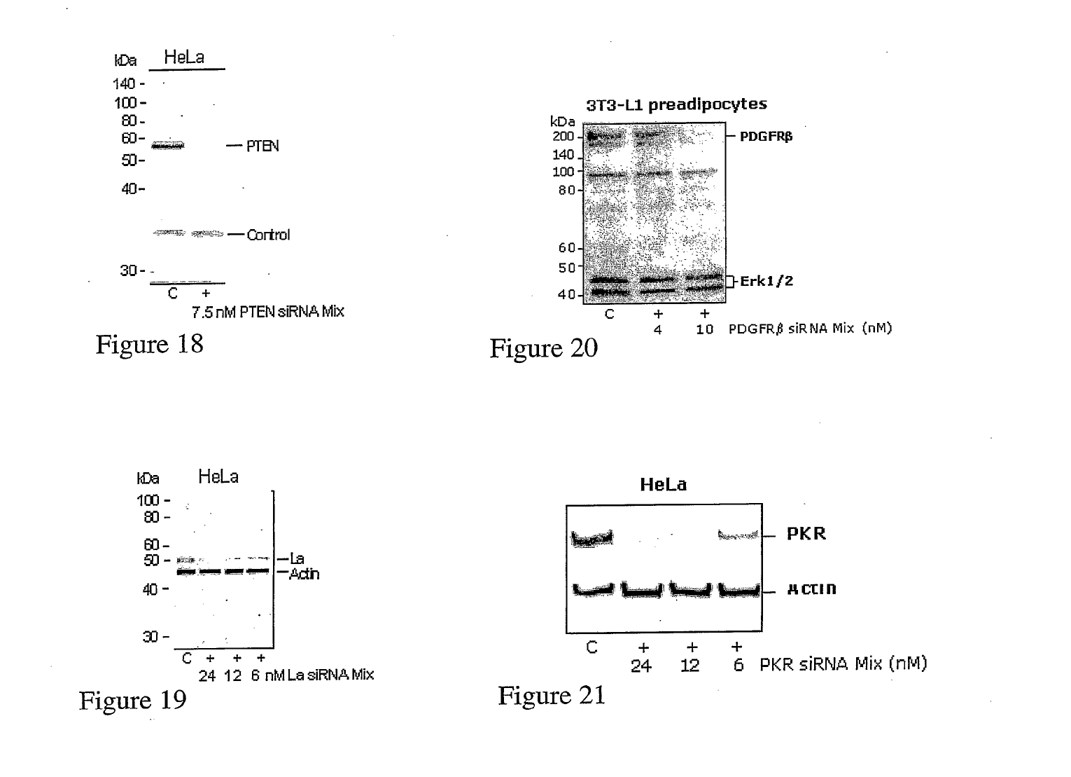

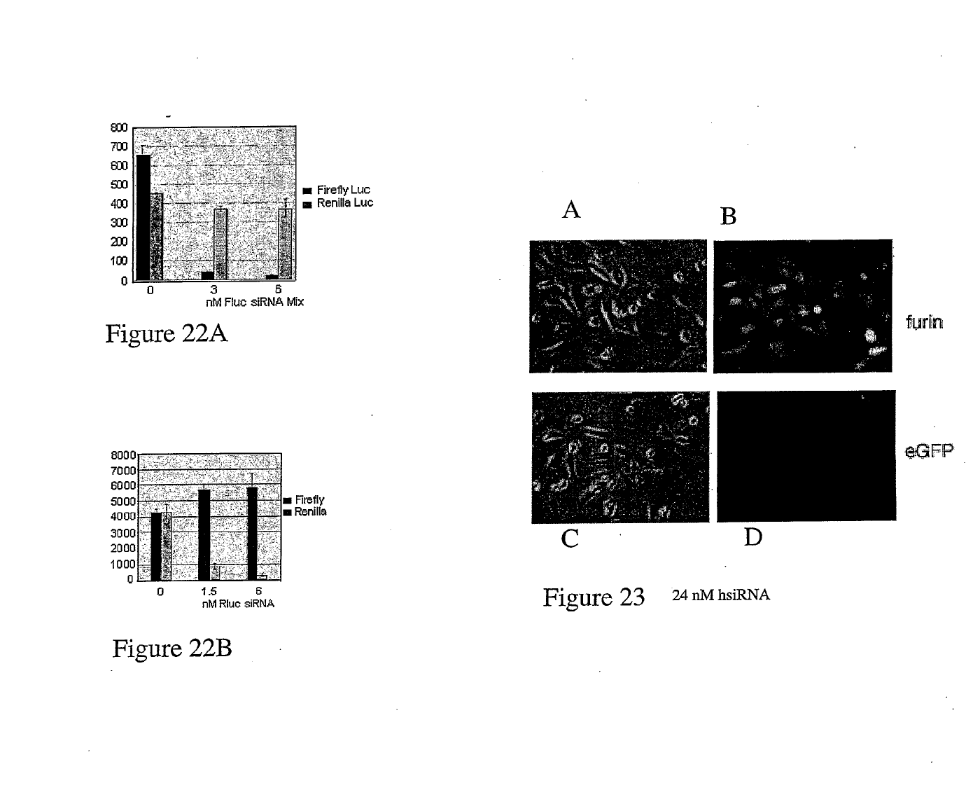

FIG. 6 shows QPCR analysis of the p53 mRNA levels in cells after transfection of 10 nM of each of 11 hsiRNA mixtures in FIG. 1, demonstrating that some hsiRNA mixtures derived from specific DNA segments are highly potent in gene silencing compared to other mixes. The three highly potent hsiRNA mixtures target non-contiguous segments (FIG. 1).

FIG. 7 shows that hsiRNA mixtures transfected in HeLa cells do not elicit the production of interferon (IFN). IFN-beta was measured with ELISA (3) in cell culture medium harvested (2) from transfected cells (1) with 20 nM hsiRNA or an equivalent amount of synthetic siRNA, or an in vitro transcription synthesized hairpin known to elicit an IFN response (+ve control)

FIG. 8 shows that the silencing effectiveness of an hsiRNA mixture that is the product of RNaseIII/Mn is greater than an hsiRNA mixture made by Dicer.

FIG. 8A shows a schematic of a reporter vector in which the promoter is cytomegalovirus (CMV), the reporter is luciferase and the target sequence is a 990 bp fragment. The transcription product of the reporter vector encodes luciferase and includes the target sequence. The amount of mRNA can be monitored by measuring luciferase activity. The mRNA levels depend on whether an hsiRNA preparation targets the 990 bp fragment and thereby destroys the message.

FIG. 8B shows luciferase activity following cotransfection of COS cells with the reporter vector and varying concentrations of an hsiRNA mixtures or Dicer product.

FIG. 9 demonstrates that hsiRNA mixture compared to two different mRNAs having 82% sequence identity do not cause cross silencing (off target effects).

FIG. 9A: Sequence alignment for Homo sapiens mitogen-activated protein kinase 1 (Erk2) labelled “Query” and Homo sapiens mitogen-activated protein kinase 3 (Erk1) labelled “Sbjct” showing the targeted region (SEQ ID NOS:12 and 13, respectively).

FIG. 9B shows a Western Blot in which amounts of Erk 1 and Erk 2 protein are determined after treatment of cells with Erk2 hsiRNA mixture. Lane 1=marker, lane 2=no hsiRNA mixture, lane 3 =2 nM hsiRNA mixture, lane 4=5 nM hsiRNA mixture and lane 5=20 nM hsiRNA mixture.

FIG. 9C shows that knowdown of Erk1or Erk2 mRNA occurs using low concentrations of corresponding hsiRNA mixtures determined by QPCR.

FIG. 9D shows that knockdown is specific and cross-silencing between Erk1and Erk2 is minimal.

FIG. 10 shows Western blot of lysates from Hela cells transfected with hsiRNA mixtures targeting Akt-1 mRNA and showing a gene silencing effect.

HeLa cells were transfected with 15 nM mus musculus thymona viral proto-oncogene 1 (Akt1) hsiRNA (+) or 15 nM Lit 28i Polylinker control hsiRNA (C) using TransPass™ R1 siRNA transfection reagent NEB #M2551S (New England Biolabs, Inc., Beverly, Mass.). Akt1/2/3 was detected in a Western blot of cell lysates using Cell Signaling Technology (CST #9272, Beverly, Mass.) antibody and actin (loading control) was detected using Sigma A-2066 Antibody (Sigma-Aldrich, St. Louis, Mo.).

FIG. 11 shows a Western blot of cell lysates from COS-7 cells transfected with hsiRNA mixtures targeting Caspase 3 mRNA and showing a gene silencing effect.

COS-7 cells were transfected with 7.5 nM Caspase-3 hsiRNA (+) or 15 nM Lit 28i polylinker control hsiRNA (C) using TransPass™ R1 siRNA transfection reagent (NEB #M2551S, New England Biolabs, Inc., Beverly, Mass.). Caspase-3 was detected with a specific antibody from Cell Signaling Technology (CST #9668, Beverly, Mass.) and Actin (loading control) with an antibody from Sigma A-2066 (Sigma-Aldrich, St. Louis, Mo.).

FIG. 12 shows a Western blot of cell lysates from HeLa cells transfected with hsiRNA mixtures targeting CREB mRNA and showing a gene silencing effect.

HeLa cells were transfected with CREB hsiRNA (+) or control synthetic siRNA (C) using TransPass™ R1 siRNA Transfection Reagent (NEB #M2552S, New England Biolabs, Inc., Beverly, Mass.). CREB was detected using Cell Signaling Technology (CST #9192, Beverly, Mass.) anti-CREB antibody and AKT (loading control) was detected using Cell Signaling Technology (CST #9272, Beverly, Mass.) antibody.

FIG. 13A shows a histogram of relative expression of DNA methyltransferase (DNMT1) in HCT116 cells determined by QRTPCR at 48 hours after transfection with a DnmT1 hsiRNA mixture, compared with untreated cells using beta actin as a reference.

FIG. 13B shows a Western blot of cell lysates from carcinoma cells (HCT 116) transfected with hsiRNA mixtures targeting Dnmt-3 mRNA and showing a gene silencing effect.

HCT 116 cells were transfected with 15 nM of Dnmt3B hsiRNA (+) or 15 nM eGFP hsiRNA as a negative control (C) using TransPass™ R1 siRNA Transfection Reagent (NEB #M2551S, New England Biolabs, Inc., Beverly, Mass.). Dnmt3B was detected in using an antibody raised against Dnmt3B.

FIG. 14 shows a Western blot of cell lysates from COS-7 cells transfected with hsiRNA mixtures targeting Rb mRNA and showing a gene silencing effect.

COS-7 cells were transfected with different concentrations of Rb hsiRNA (+) or 24 nM eGFP control hsiRNA (−) using TransPass™ R1 siRNA Transfection Reagent (NEB #M2551S, New England Biolabs, Inc., Beverly, Mass.). Rb was detected using a specific antibody from Cell Signaling Technology (CST #9309, Beverly, Mass.). Actin (loading control) was detected using (Sigma A-2066) antibody (Sigma-Aldrich, St. Louis, Mo.).

FIG. 15 shows a Western blot of cell lysates from NIH3T3 cells transfected with hsiRNA mixtures targeting mitogen stress activated kinase (MSK1) and showing a gene silencing effect.

NIH3T3 cells were co-transfected with a plasmid expressing MSK1 tagged with an HA epitope and different concentrations of MSK1 Kinase hsiRNA (+) or Lit28i Polylinker hsiRNA using TransPass™ R1 Transfection Reagent (NEB #M2551S, New England Biolabs, Inc., Beverly, Mass.). MSK1 was detected 48 hours post-transfection using Cell Signaling Technology anti-HA Antibody (CST #2367, Beverly, Mass.). Actin (loading control) was detected using Sigma (#A2066) antibody (Sigma-Aldrich, St. Louis, Mo.).

FIG. 16A shows a Western blot of cell lysates from U2OS cells transfected with hsiRNA mixtures targeting estrogen receptor alpha (ERalpha) mRNA and showing a gene silencing effect.

U2OS cells stably expressing inducible alpha estrogen receptor (ERalpha) were mock-transfected or transfected with different concentrations of ERalpha hsiRNA (+). ERalpha was detected 48 hrs post transfection using DakoCytomation (M7047, Carpinteria, Calif.) anti-ER alpha antibody and actin (loading control) was detected with anti-actin antibody.

FIG. 16B shows a Western blot of cell lysates from U2OS cells transfected with hsiRNA mixtures targeting estrogen receptor beta (ERbeta) mRNA and showing a gene silencing effect.

U2OS cells stably expressing inducible estrogen receptor beta were transfected with different concentrations of ERbeta hsiRNA (+) or 80 nM of ERbeta hsiRNA as a negative control (+) as indicated, using TransPass™ R1 Transfection Reagent (NEB #M2551S, New England Biolabs, Inc., Beverly, Mass.). (−) indicates a mock transfection with Transpass™ R1 Transfection Reagent only. ERbeta was detected 48 hr post transfection using anti-ERbeta monoclonal antibody (GeneTex Inc., San Antonio, Tex.). Actin (loading control) was detected using anti-actin antibody (Oncogene Research Products, now Merck Biosciences, Nottingham, UK).

FIG. 17A shows a Western blot of cell lysates from HeLa cells transfected with hsiRNA mixtures targeting p38 MAPK mRNA and showing a gene silencing effect.

HeLa cells were transfected with different concentrations of p38 MAPK hsiRNA (+) or 24 nM enhanced green flourescent protein (eGFP) control hsiRNA Mix (C) using TransPass™ R2 siRNA Transfection Reagent (NEB #M2552S, New England Biolabs, Inc., Beverly, Mass.). p38 MAPK was detected 48 hours post-transfection using anti-p38 antibody (CST #9217, Cell Signaling Technology, Beverly, Mass.) and human p68 kinase (PKR) (loading control) was detected using CST #3072 (Cell Signaling Technology, Beverly, Mass.) antibody.

FIG. 17B shows a Western blot of cell lysates from COS-7 cells transfected with hsiRNA mixtures targeting Erk2 mRNA and showing a gene silencing effect.

COS-7 cells were transfected with 6.0 nM Erk2 Kinase hsiRNA (+) or 15 nM Lit 28i Polylinker control hsiRNA (C) using TransPass™ R1 siRNA transfection reagent (NEB #M2551S, New England Biolabs, Inc., Beverly, Mass.). Erk2 was detected 48 hours post-transfection using anti ERK1/2 antibody (Cell Signaling Technology, Beverly, Mass., CST #9107,) and PKR (loading control) was detected using CST #3072 (Cell Signaling Technology, Beverly, Mass.) antibody.

FIG. 18 shows a Western blot of cell lysates from HeLa cells transfected with hsiRNA mixtures targeting phosphatase and tensin homolog (mutated in multiple advance cancers 1) (PTEN) mRNA and showing a gene silencing effect.

HeLa cells were transfected with 7.5 nM PTEN hsiRNA (+) or 15 nM Lit28i Polylinker hsiRNA (C) using TransPass™ R1 siRNA transfection reagent (NEB #M2551S, New England Biolabs, Inc., Beverly, Mass.). PTEN was detected using anti-PTEN antibody (Cell Signaling Technology, Beverly, Mass., CST #9552) 48 hours post-transfection.

FIG. 19 shows a Western blot of cell lysates from HeLa cells transfected with hsiRNA mixtures targeting La mRNA and showing a gene silencing effect.

HeLa cells were transfected with different concentrations of La hsiRNA (+) or 24 nM eGFP control hsiRNA (C) using TransPass™ R2 siRNA Transfection Reagent (NEB #M2552S, New England Biolabs, Inc., Beverly, Mass.). La was detected with anti-La Antibody (Transduction Laboratories, now BD Biosciences, San lose, Calif. #9610804) and actin (loading control) was detected using A-2066 antibody (Sigma-Aldrich, St. Louis, Mo.).

FIG. 20 shows a Western blot of cell lysates from 3T3-L1 preadipocyte cells transfected with hsiRNA mixtures targeting platelet derived growth factor receptor (PDGFR) beta mRNA and showing a gene silencing effect.

3T3-L1 preadipocytes were transfected with different concentrations of PDGFR beta hsiRNA (+) or 30 nM Lit28i Polylinker hsiRNA as a control (C) using Transpass™ R1 siRNA Transfection Reagent (NEB #M2551S, New England Biolabs, Inc., Beverly, Mass.). PDGFR beta was detected 72 hours post-transfection using anti-PDGFR beta antibody from Upstate-Cell Signaling Solutions, now Serologicals Corporation, Norcross, Ga. (#06-498) and Erk1/2 (loading control) was detected using antibody from Cell Signaling Technology (CST #9102, Beverly, Mass.).

FIG. 21 shows a Western blot of cell lysates from HeLa cells transfected with hsiRNA mixtures targeting PKR mRNA and showing a gene silencing effect.

HeLa cells were transfected with different concentrations of PKR hsiRNA (+) or 24 nM eGFP control siRNA Mix (NEB #N2015S, New England Biolabs, Inc., Beverly, Mass.) (C) using TransPass™ R2 siRNA transfection reagent (NEB #M2552S, New England Biolabs, Inc., Beverly, Mass.). PKR was detected using anti-PKR antibody Cell Signaling Technology (CST #3072, Beverly, Mass.) and actin (loading control) was detected using A-2066 Antibody (Sigma-Aldrich, St. Louis, Mo.).

FIG. 22A is a histogram showing specific silencing of firefly luciferase (Fluc). Bars show Renilla (light bars) and Firefly luciferase (dark bars) activities from cells transfected with Fluc expressed in Relative Luminescence Units (RLU).

COS-7 cells were co-transfected with Firefly (GL3) and Renilla (pRLTK) luciferase expression plasmids (Promega Corporation, Madison, Wis.) and with the indicated concentrations of Fluc hsiRNA.

FIG. 22B is a histogram showing specific silencing of Renilla Luciferase Bars show Renilla (light bars) and Firefly Luciferase (dark bars) activities from cells transfected with hsiRNA directed Renilla luciferase against expressed in Relative Luminescence Units (RLU).

U20S cells were transfected with a Firefly (GL3) and a Renilla (pRLTK) Luciferase expression plasmids (Promega Corporation, Madison, Wis.) and with the indicated concentrations of Renilla luciferase hsiRNA.

FIG. 23 are microphotographs of U2OS cells transfected with eGFP hsiRNA showing silencing of eGFP.

U2OS cells stably transfected for eGFP expression were transfected with 24 nM hsiRNA for eGFP (FIGS. 23C and 23D) or for furin as a negative control (FIGS. 23A and 23B). Live transfected cells were photographed 48 hrs post transfection in the visible channel (FIGS. 23A and 23C) or fluorescence channel (FIGS. 23B and 23D).

### DETAILED DESCRIPTION

It is desirable to maximize the potency and efficacy of mixtures of hsiRNA for gene silencing. Highly potent gene silencing mixtures of overlapping fragments of dsRNA of a size between about 18-30 nt are described herein which have been selected by assaying multiple hsiRNA mixtures in efficient and reproducible biological assays. Each hsiRNA mixture is produced efficiently by wild type (wt) RNaseIII or mutant RNaseIII cleavage from dsRNA templates having sequence identity for different regions of the mRNA.

The following terms as used in the description and in the accompanying claims have been defined below. These definitions should be applied unless the context in which the terms are used requires otherwise.

“hsiRNA mixture” refers to a heterogeneous (h) mixture of short double-stranded RNA fragments containing at least one fragment (siRNA) suitable for silencing gene expression. The RNA fragments in the hsiRNA mixture consistently contain a substantial fraction (greater than about 15% of the total number of fragments) having a length of 18-30 base pairs as determined by ethidium-stained native polyacrylamide gel analysis.

“Silencing” refers to partial or complete loss-of-function through targeted inhibition of gene expression in a cell and may also be referred to as “knock down”.

“Large double-stranded RNA” refers to any double-stranded RNA having a size greater than about 40 base pairs (bp), for example, larger than 100 bp or more particularly larger than 300 bp. The sequence of a large dsRNA may represent a segment of a messenger RNA (mRNA) or the entire mRNA. The maximum size of the large dsRNA is not limited herein. The dsRNA may include modified bases where the modification may be to the phosphate sugar backbone or to the nucleoside. Such modifications may include a nitrogen or sulfur heteroatom or any other modification known in the art. The dsRNA may be made enzymatically, by recombinant techniques and/or by chemical synthesis or using commercial kits such as MEGASCRIPT® (Ambion, Austin, Tex.) and methods known in the art. An embodiment of the invention utilizes HiScribe™ (New England Biolabs, Inc., Beverly, Mass.) for making large dsRNA. Other methods for making and storing large dsRNA are described in International Publication No. WO 99/32619.

The double-stranded structure may be formed by self-complementary RNA strand such as occurs for a hairpin or a micro RNA or by annealing of two distinct complementary RNA strands.

“Heterogeneous” in the context of an hsiRNA mixture refers to dsRNA fragments having non-identical sequences produced from a single large dsRNA or a mixture of large dsRNAs after cleavage with RNaseIII in the presence of manganese ions or with mutant RNaseIII in a magnesium or manganese buffer. The fragments collectively contain sequences from the entire length of the large RNA and hence form a heterogeneous mixture.

“RNaseIII” refers to a naturally occurring enzyme or its recombinant form and may include mutants and derivatives or homologs. The utility of bacterial RNaseIII described herein to achieve silencing in mammalian cells supports the use of RNases from eukaryotes or prokaryotes in the present embodiments. Embodiments of the invention do not preclude the use of more than one RNase to prepare an hsiRNA mixture. RNaseIII as defined here is characterized by an amino acid consensus sequence in the protein [DEQ]-[kRQT]-[LM]-E-[FYW]-[LV]-G-D-[SARH] (PROSITE: PDOC00448 documentation for the RNaseIII).

The use of highly potent gene silencing mixtures overcomes the problems of high cost and labor-intensive design and synthesis of individual effective siRNA reagents and the associated non-specific “off target” effects at the standard concentration of effectiveness and provides a means to maximize the number of silencing experiments possible from a small amount of an hsiRNA mixture.

Present embodiments of the invention include the finding that certain hsiRNA mixtures that have specificity for certain regions of an mRNA have significantly greater gene silencing activities than mixtures that are specific for other regions. These hsiRNA mixtures are described as “highly potent”. FIG. 6 shows how certain hsiRNA mixtures are as much as about twice as potent as other hsiRNA mixtures (see for example: C20 hsiRNA mixture compared to C9 siRNA mixture in FIG. 6).

Other embodiments of the invention include the increased activity of hsiRNA mixtures over Dicer siRNA where less than 1 nM hsiRNA effectively silences a gene as detected by a luciferase assay in contrast to 4 nM of Dicer siRNA to gain a similar effect.

The use of lower concentrations of an hsiRNA mixture or an siRNA to achieve gene silencing is advantageous because the lower concentration of any particular fragment in the mixture results in lower or no undesirable off-target effects. Other advantages of using low amounts of any reagent are reduced cost and increased convenience.

Another embodiment of the present invention is the ability to achieve gene silencing with high specificity. This is demonstrated in FIG. 9 in which an hsiRNA mixture is observed to discriminate between two genes having highly related sequences. For example, one of Erk1 and Erk2, which together have more than 80% sequence identity, can be selectively silenced by a specific hsiRNA. FIG. 9 shows how increasing amounts of Erk2 hsiRNA mixtures from 160 pM to 800 pM results in increasing gene silencing (% remaining RNA) of Erk2 but not of Erk1. Similarly, increasing amounts of Erk1 hsiRNA mixtures from 160 pM to 800 pM increases silencing of Erk1 but not Erk2 (FIG. 9C). Specificity is also observed when high concentrations of hsiRNA mixtures are also used up to 50 nM.

Advantages hsiRNA mixtures described herein or made according to methods described herein include:

(a) Ease of preparation of the hsiRNA mixture to provide an enhanced concentration of dsRNA fragments of a size suitable for silencing of gene expression by a rapid, cost effective process that is not dependent on a gel based size separation step (also see US Publication No. 2004-0038278).

(b) Enzymatic digestion of large dsRNA means that a particular 18-30 nt fragment does not have to be selected for synthesis but rather a longer stretch of sequence can serve as a substrate for enzyme digestion to form multiple overlapping fragments having a size suitable for gene silencing (see FIGS. 1 and 2).

(c) The ease of providing gene-silencing reagents facilitates studies on expression of families of genes or metabolic pathways

(d) Any of the hsiRNA reagents described herein or that are the product of methods described herein may be used as therapeutic agents or in therapeutic agent screening or target validation assays.

(e) Where use of very low concentrations of any siRNA is desired, regions of the gene can be identified for which hsiRNA mixtures can be prepared to provide highly potent silencing activity (FIG. 6 and 8).

(f) The compositions and methods described herein avoid enhancing IFN expression (FIG. 9)

Highly potent mixtures of the sort described herein provide gene silencing with a high degree of specificity, lower cost, and the ability to multiplex assays without saturating the cells.

Highly potent hsiRNA mixtures can be made for any target gene of interest. This general observation has been exemplified by p53, Erk1,2 (FIG. 9) and luciferase (FIG. 22) but is not intended to be limited to the same. For example, 3 of 11 hsiRNA mixtures spanning about 2400 nt of p53 had high potency and efficacy for gene silencing. Indeed, transfection concentrations of less than 1 nM for an hsiRNA mixture derived from a 900 bp fragment (FIG. 8) were sufficient to cause more than 70% silencing. FIG. 8 shows that concentrations of 0.01-0.1 nM of the hsiRNA mixture caused about 70% silencing. The high potency of these mixtures results in the use of low concentrations of dsRNA fragments which in turn avoids both general and sequence-related non-specific effects.

Highly potent mixtures can be obtained by preparing hsiRNA mixtures against one or more similar or different sized regions of the target sequence, where different sizes are, for example, from 60 to 1000 nt long (FIG. 1). Although preparations with overlapping sequences of the sort generated by RNaseIII in the presence of manganese ions or by mutant RNaseIII provide the desired range of fragments that reduce non-desirable effects, it is nevertheless possible to cleave a large dsRNA with other enzymes such as Dicer or Drosha or with wt RNaseIII under magnesium salt conditions or with a chimeric enzyme or engineered nuclease in order to prepare a highly potent siRNA mixture. The potency of the siRNA mixtures prepared from single targets or multiple contiguous or non-contiguous targets from a single messenger RNA can then be determined.

It is here proposed that complexity in the mixture, meaning the number of siRNA fragments as well as the degree of overlap and representation of the current sequence, enhances gene silencing effects while reducing the undesirable off-target effects.

FIG. 8 shows how an RNaseIII digest (with manganese) which creates overlapping fragments (US Publication No. 2004-0038278) results in a more potent preparation than that observed with Dicer which cuts sequentially along a dsRNA.

The potency of hsiRNA mixtures can be determined by cell assays including high throughput assays to show actual down-regulation of gene expression. For example, a population of cells can be transfected at low concentrations and the level of gene silencing determined by protein or mRNA amounts. This can be done by Western blots, QPCR, RT PCR, measuring enzymatic activity such as luciferase by fluorescence, in situ hybridization, cell sorting or any other detection method known in the art. (Examples are shown in FIGS. 5-10.)

The extent of gene silencing as determined by the amount of mRNA extracted from the cell population is, unless further qualified, a combination of efficiency of gene silencing in a single cell and the efficiency of transfection of the cell population.

The extent of gene silencing as determined by the amount of protein measured in a cell population is not only a product of efficacy of silencing in a single cell and the efficient transfection in the population but it is also the efficacy of protein translation of the message that is produced as well as the sensitivity of detection methods. In the above context, the efficacy of knockdown (gene silencing) of a population of cells after transfection with less than 2 nm of an hsiRNA mixture is at least 65% in a transfected cell, for example, 70% or more, particularly 75% or 80% in transfected cells. Knock-down levels of as much as or more than 90% were obtained when using the hsiRNA mixtures for p53 prepared from the templates with the non-contiguous pieces C6, C14, C20 (FIGS. 1 and 6).

COS-7 cells have been used as a standard cell. For these cells, transfection efficiency may be as high as 85% to 95%. Different cells may vary widely in terms of transfection efficiency in a manner that is well known in the art. For example, transfection efficiencies can vary depending on whether the target cells are transformed or are primary cells or are in vivo tissues and depending on whether transfection is achieved by chemical means or viral vectors.

In the Examples and Figures, p53, Renilla luciferase and Erk1,2 genes were used as a target for gene silencing to demonstrate this method. Similar results can be obtained for any selected sequence.

The following characteristics are demonstrated here for gene silencing:

(a) sequences along the length of the mRNA can be used as targets for gene silencing although some sequences result in greater gene silencing than others.

(b) the size of the dsRNA that is used to form hsiRNA mixtures is not critical to gene silencing activity.

(c) mixtures of hsiRNA preparations from non-contiguous regions of the mRNA can be highly potent.

(d) off-target effects are minimized using hsiRNA mixtures because any individual sequence in a mixture of overlapping sequences is present at very low concentrations. This can be calculated as follows:

The maximum possible number of different siRNAs of a size of 22 nt in an hsiRNA mixture from a given sequence of length L nucleotides is (22×L)−22. The theoretical concentration fraction of each siRNA in the mixture from that sequence is [22×(L−1)]⁻¹. Thus the fraction of each individual siRNA in the mixture proportionally decreases as the length of the fragment increases. For example the concentration of each individual siRNA in 10 nM of a mixture obtained from a 66 bp fragment is [(22×65)]⁻¹ ×10=6.99 pM. This concentration becomes 0.75 pM for siRNAs from a fragment of 600 bp.

Unless otherwise defined, the terms used herein are intended to have the meanings put forth in US Publication No. 2004-0038278 incorporated herein by reference.

All references recited herein are incorporated by reference as well as provisional applications 60/543,880 and 60/551,558 and PCT application PCT/US 05/02029.

### EXAMPLES

### Example 1

### Preparation of Different hsiRNA Mixtures Against the Same Target Gene

1. Preparation of DNA Transcription Templates

An hsiRNA mixture from human p53 CDNA was used to silence monkey p53 gene expression. It was first established that the percentage sequence identity between human and monkey p53 genes was about 98%. p53 template DNA was prepared by (a) cloning or (b) PCR amplification as follows:

(a) p53 clones: A PCR-generated p53 CDNA fragment was digested with AciI (New England Biolabs, Inc., Beverly, Mass.) and the mixture of fragments was ligated into Litmus 28i plasmids (New England Biolabs, Inc., Beverly, Mass.) digested with BstBI (New England Biolabs, Inc., Beverly, Mass.). The engineered plasmids were used to transform E. coli. After transformation, single colonies were selected and analyzed by DNA sequencing. After sequencing the cloned p53 inserts, their position on the p53 human cDNA sequence was determined by BLAST 2 sequences alignment.

(b) Three sets of primers were designed for use in amplifying those regions of the p53 sequence that had not been cloned (Table 1). Each primer contained the T7 promoter sequence and the forward primers additionally had a biotin molecule attached to the 5′ end.

As shown in FIG. 1, the complete 2.6 kb sequence of p53 mRNA was covered by cloned or amplified fragments except the first 128 and the 60 last nucleotides (FIG. 1).

The size of each cloned or amplified fragment varied from a minimum of 66 to approximately 600 bp. Some of the clones (C6, C9, C14, C20) contained non-contiguous segments of the p53 sequence.

DNA templates for in vitro transcription from Litmus 28i clones (C-constructs) (FIG. 1) were generated by PCR with biotinylated T7 primer: 5′-Biotin-d (CTCGAGTAATACGACTCACTATAGG) 3′ (SEQ ID NO:1). PCR was performed with 1 to 10 ng of each construct's plasmid DNA. p53 templates were obtained by using these primers to perform standard PCR on total cDNA from HEK293 or COS-7 cells. The same annealing temperature (68° C.) for the three sets of primers was used.

After each PCR, a 1% agarose gel was used to check the size of each fragment and the PCR efficiency.

2. Purification of DNA Transcription Templates

Streptavidin-coated magnetic beads were used to purify PCR products described above in the following steps: adjustment of each PCR reaction at 0.5 NaCl, addition of 50 μL of beads in each PCR reaction and binding on the beads for 15 min, 3 washes (with TE [10 Mm Tris-Hcl pH 8, 1 mM EDTA] 0.5M NaCl first and then with TE only) and resuspension in 20 μL of TE buffer. The PCR fragments immobilized on the beads were used directly for in vitro transcription.

3. In vitro Transcription to Make dsRNA

Transcription reactions were performed in 60 μL of transcription mix (Hiscribe™ kit, New England Biolabs, Inc., Beverly, Mass.) according to the kit instructions to produce dsRNA from each strand of the DNA transcription template: After 2 h incubation at 42° C., an annealing protocol was performed for 3 min at 72° C., followed by 3 min at 68° C., 5 min at 65° C., 10min at 62° C. and a final step at 42° C. for 15 min. Transcription and annealing was performed in a PCR machine. The magnetic beads were removed by magnetic separation and the mixtures were diluted two-fold with water, then adjusted to 0.3 M sodium acetate pH 5.2, and precipitated with 2 volumes of cold 95% ethanol overnight at −20° C. After 15 min of centifugation at 14000 rpm, the pellet was washed once with 70% ethanol, allowed 15 min to air dry and resuspended in 50 μL of water. The dsRNA was dissolved by 5 min incubation at 70° C. and 10 min at room temperature.

4. Preparing hsiRNA Mixtures

Before the digestion, the concentration of dsRNA produced according to FIG. 2A was determined on 1% agarose gel (FIG. 2B) by comparison to DNA standards by using a suitable software package (Quantity One™ from Bio-Rad Laboratories, Hercules, Calif.). To prepare hsiRNA, 10 μg of dsRNA were digested in a 100 μL reaction buffer using Shortcut™ RNaseIII kit (New England Biolabs, Inc., Beverly, Mass.) and following kit instructions. After ethanol precipitation in the presence of 20 μg of glycogen, the samples were resuspended in 30 μL of water. 10% of each digestion was loaded on a non-denaturing TBE 20% polyacrylamide gel to verify and quantify the digestion products by comparison to a 21 bp synthetic siRNA standard marker. (An example is shown in FIG. 2C.)

For hsiRNA mixtures, molarity was calculated based on the average molecular weight of the dsRNA which is: 2×22×361=15884 g/mol (361 is the average molecular weight of the 4 RNA bases). For example, a molarity of 10 nM in a transfection performed in 0.3 mL (24 wells plate) corresponds to 4765 ng per transfection well, where: Xng×(1 nmol/15884 ng)×1/0.3×10^{-'}L=10 nM where X=4765 ng per transfection well.

### Example 2

### Testing of Relative Effectiveness (Potency) of hsiRNA Mixtures

Cell Culture and hsiRNA Transfections

COS-7 cells were cultured using standard techniques and passaged every three days and twenty-four hours before transfection with a hsiRNA mixture. Transfection with different amounts of hsiRNA was performed using Transpass™ RI transfection reagent (New England Biolabs, Inc., Beverly, Mass.) according to the manufacturer's instructions. Cells were transfected at 50-70% confluence in 12 or 24-well plates containing respectively 1 mL or 0.5 mL of medium per well. Transfection reagent (3 μL/well for 24-well plates) was mixed with serum-free media (50 μL) and incubated for 10 min. Complete fresh growth medium (250 μL) was then added to each tube and 300 μL were aliquoted in each well after aspirating the old medium from each well. Cells were grown at 37° C. for 48 hours, washed two times with cold BPS and analyzed as described below.

Transfection efficiency was determined by fluorescence microscopy of cells transfected with a fluorescein-labeled synthetic siRNA (New England Biolabs, Inc., Beverly, Mass.).

Knockdown of Protein Expression-Western Blots Cells were lysed on ice in 1% Triton-X, 50 mM Tris-HCl, 150 mM NaCl (Lysis Buffer) and supernatants were stored at −20° C. after centrifugation. Equal amounts of protein (10 μg) from each transfected well (determined by BCA protein assay (Pierce BioTechnology, Rockford, Ill.) was resolved by 10-20% polyacrylamide Tris-glycine gel (Invitrogen Corporation, Carlsbad, Calif.) in SDS buffer (125 mM Tris, 1 M glycine, 20 mM SDS) at 30 mA constant for 1 hour. A pre-stained protein marker and a biotinylated marker were used to monitor protein migration, blotting of protein and for size determination. The gels were blotted on to a PVDF membrane (Millipore Immobilon-P™ transfer membrane 0.45 μm, Millipore, Billerica, Mass.) and stained with 0.1% Ponceau Red (Sigma-Aldrich, St. Louis, Mo.). The membrane was blocked in Blocking buffer (1× TBS: 20 mM Tris base, 136 mM NaCl; 0.15% Tween; 5% w/v non-fat dry milk). After blocking, two different antibodies were used: a mouse monoclonal anti-p53 (Cell Signaling Technology, Beverly, Mass.) diluted 1/1000 and a rabbit polyclonal anti-actin (Sigma-Aldrich, St. Louis, Mo.) dilution 1/3000 as a control. Immune complexes were visualised with the Phototope-HRP (horseradish peroxidase) Western Blot Detection System containing the anti-rabbit HRP-conjugated secondary antibodies, a biotinylated marker and the detection reagent LumiGlo (Cell Signaling Technology, Beverly, Mass.). When necessary stripping of the membrane (Restore Western Blot Stripping Buffer (Pierce Technology, Rockford, Ill.) was performed after the detection of p53. The membrane was then reprobed with anti-actin or other antibodies. The results are shown in FIG. 3.

### Knockdown of RNA-QPCR

### Preparation of Total RNA

The kit RNA Aqueous™ (Ambion, Inc., Austin, Tex.) was used to prepare total RNA from crude cell extracts according to kit instructions. Before cDNA synthesis, the quality and amount of RNA was analyzed by 1% agarose gel electrophoresis and by spectrophotometry

### First Strand CDNA Synthesis

The synthesis of cDNA was performed in 20 μL reactions according to the instructions of the Protoscript™ first strand cDNA synthesis kit (New England Biolabs, Inc., Beverly, Mass.) using approximately 0.5 to 1 μg of the extracted total RNA and the reaction was diluted to 50 μL in water.

After each synthesis, a standard PCR with 0.2 μM Glyceraldehyde-3-phosphate dehydrogenase (GAPDH) primers was performed to verify the efficiency of synthesis.

Real-Time PCR Assays

Different sets of specific primers were designed and optimized for p53 and beta-actin. The annealing temperature was 60° C. The sequences of these primers and their position on each cDNA sequence are described in Tables 1 and 2.

The amplification reactions were performed in 50 μL total volume containing 1× final iQ SYBR green supermix™ (BioRad Laboratories, Hercules, Calif.) according to kit instructions and 2 μL (1/25 of total cDNA volume) of cDNA.

A master mix was prepared for each set of primers and aliquoted in each well, prior to the addition of the DNA samples. The plate was pulse-centrifuged at 1000 rpm. The following PCR program was performed on the i-Cycler Thermal Cycler™ (BioRad Laboratories, Hercules, Calif.): 95° C. for 5 min, 30 sec 95° C., 30 sec 60° C. for 40 to 50 cycles, 95° C. for 1 min. The data for the melting curves were acquired every 0.5° C. from 40° C. to 95° C. (110 steps).

The results were expressed using the threshold cycle (Ct), which was set at the point in which the signal generated from the sample is significantly greater than background fluorescence. The knock-down of p53 was evaluated relative to beta-actin as the reference gene (FIG. 4). The linear detection range and reproducibility of the assay was assessed from the standard curves generated for both p53 and beta actin using serially diluted cDNA and triplicate samples. The PCR efficiency was 100.1% and the curve's correlation coefficient (r²) 0.995.

The formula used for each sample was: 2^{(-(Ct p53-Ct actin))} where Cₜ p53 is the threshold cycle for amplification of p53 and Cₜ actin is the threshold cycle for actin (Heil et al. BioTechniques 35:502-508: 2003). The knock-down efficiency was calculated as a fraction of the relative expression of RNAi samples (from cells transfected with a siRNA mixture targeting p53 mRNA) to relative expression of the control (cells transfected with a siRNA mixture targeting another mRNA, CREB) which was set to 100%. The different mixtures were studied several times with an average standard deviation of 3.6%. FIGS. 3, 5 and 6 show the results of knockdown of p53 after transfection with hsiRNA as determined by measuring decrease in transcription product (relative expression %).

TABLE 1

PCR primers designed from human p53 cDNA sequence The
coordinates for the position of each amplified fragment are
shown on the human p53 cDNA sequence (gi 8400737) in nucleo-
tide number (B = biotin)

| Name and position | |
|---|---|
| on human p53 cDNA | |
| sequence | Designed Primers |
| P 2 | Forward primer |
| 128-712 | 5′- |
| | BCTCGAGTAATACGACTCACTATAGGCCGTCCAGGGAGCAGGTA |
| | GCT-3′ |
| | (SEQ ID NO: 2) |
| | Reverse primer |
| | 5′- |
| | CTCGAGTAATACGACTCACTATAGGCCGGGCGGGGGTGTGGAAT |
| | C-3′ |
| | (SEQ ID NO: 3) |
| P 3 | Forward primer |
| 1428-2021 | 5′- |
| | BCTCGAGTAATACGACTCACTATAGGGACTGACATTCTCCACTTC |
| | TT-3′ |
| | (SEQ ID NO: 4) |
| | Reverse primer |
| | 5′- |
| | CTCGAGTAATACGACTCACTATAGGCTCTACCTAACCAGCTGCCC |
| | AACT-3′ |
| | (SEQ ID NO: 5) |
| P 4 | Forward primer |
| 1968-2567 | 5′- |
| | BCTCGAGTAATACGACTCACTATAGGCCTGTTGGCTGGTGGGTTG |
| | GTAGT-3′ |
| | (SEQ ID NO: 6) |
| | Reverse primer |
| | 5′- |
| | CTCGAGTAATACGACTCACTATAGGAGGAGGGGAAGGGTGGGGT |
| | GAAAA-3′ |
| | (SEQ ID NO: 7) |

TABLE 2

Primers used for real-time PCR assay and their
position on each corresponding Human and Green
African Monkey cDNA sequence.

| | |
|---|---|
| | Position on cDNA |
| | sequence and |
| | size of the |
| Primers | amplicon |

| | | |
|---|---|---|
| Beta-actin | Forward primer | Size: 98 bp |
| real-time | TGCGTGACATTAAGGAGAAG | Position: |
| PCR primers | (SEQ ID NO: 8) | Human sequence |
| | Reverse primer | (gi 5016088): |
| | GCTCGTAGCTCTTCTCCA | 699-797 African |
| | (SEQ ID NO: 9) | Green Monkey |
| | | sequence (gi |
| | | 2116654): |
| | | 636-734 |
| p 53 real- | Forward primer | Size: 197 bp |
| time PCR | CCCCCTCCTGGCCCCTGTCATC | Position: |
| primers | TT | Human sequence |
| | (SEQ ID NO: 10) | (gi 8400737): |
| | Reverse primer | 514-711 African |
| | CGGGCGGGGGTGTGGAATCAA | Green monkey |
| | C | sequence (gi |
| | (SEQ ID NO: 11) | 22795): 365-562 |

### Example 3

### Measuring Gene Silencing in Intact Cells

A Reporter Vector for Assessing Potency of hsiRNA Mixtures in Intact Cells.

We constructed a reporter vector used to easily measure the potency of hsiRNA mixtures using a secreted luciferase (G-luc) activity assay. The coding region for Gaussia princeps luciferase was expressed under the control of CMV promoter. Cloning sites in the 5′ and 3′ untranslated region were used for cloning the target sequence. A 990 bp DNA fragment was inserted in the 3′ UT region of the transcript and was targeted with different concentrations of hsiRNA mixtures generated with either RNaseIII in Mn²⁺ buffer or human Dicer digestions of a 300 bp dsRNA subfragment of the 990 bp sequence. The vector was co-transfected with different concentrations of hsiRNA mixture as described in Example 2 for HeLa cells. The luciferase activity was measured 24 hr after transfection using the Promega Renilla assay buffer. Significant silencing (>75%) of activity was achieved with the former at 0.2 nM versus 2 nM. The results are shown in FIG. 8 which compares the silencing effect of similar concentrations of hsi RNA from RNaseIII with Dicer generated siRNA.

### Example 4

### Interferon is Not Induced by hsiRNA Transfection

IFN-beta was measured with a commercial ELISA assay kit (PBL Biomedical Laboratories, Piscataway N.J.) in cell culture medium from cells transfected with 20 nM hsiRNA or an equivalent amount of synthetic siRNA, or an in vitro transcription synthesized hairpin known to elicit this response (Kim et al. Nat Biotechnol. 3:321-5 (2004)). The results are shown in FIG. 7. IFN was not produced as a result of hsiRNA transfection.

### Example 5

### Examples of hsiRNA Mixtures

HsiRNA mixtures were made and tested and found to be effective for Akt1, 2, 3, Erk1, 2, Msk 1, p38, IRS1, PKR, PTEN, CREB, ERa, ERb, DAX, p53, DNMT1, DnMT3B, DnMT3A, TRIP, Rb, MeCP2, Caspase3, La, Furin, EGFP, RFP, Ffluc and Renilla luciferase. (see FIGS. 10-23). These preparations were made to be endotoxin free

### Example 6

### Endotoxin Removal from Heterogeneous siRNA Mixture

Endotoxin levels can be significantly reduced by using Source RPC™ (Pharmacia Biotech, now GE-Healthcare, Chaifont St. Giles, UK) directly after column purification to sample levels below 1EU. The samples was loaded on a 3 ml Source RPC column.
Buffer A: 35 mM triethylamine (pH 7.0 with acetic acid at 250° C.), 2% acetonitrile made with Milli-Q or equivalent water.
Buffer B: 100% acetonitrile.
The column was run on an AKTA FPLC™ system (Pharmacia Biotech, now GE-Healthcare, Chaifont St. Giles, UK) to remove any contaminating endotoxin from the siRNA mixture which elutes at approximately 50% buffer B.
Fractions were transferred to 1.5 ml micro-centrifuge tubes and dried overnight in a spin vacuum without heat. Pellets were hydrated with storage buffer at room temperature.

### Example 7

### Absence of Off-Target Effects: Analysis of Silencing in Related but Different Genes

Quantitative real-time PCR was used to investigate the specificity of hsiRNA mixes targeting Erk2 and Erk1 having 82% sequence identity with each other along a 281 nucleotide region of their mRNAs.

HeLa cells were transfected with Transpass R1 (New England Biolabs, Inc., Beverly, Mass.) and (a) 10 nM or 50 nM Erk2 hsiRNA, or 10 or 50 nM Erk1 hsiRNA in triplicate (FIG. 9(d))
(b) 20 nM GFP hsiRNA mix, 160 pM, 800 pM, 4 nM Erk2 hsiRNA mix or 160 pM, 800 pM, 4 nM Erk1 hsiRNA mix (FIG. 9c)
   where the hsiRNA mixtures were made according to Example 1 (4).

Cells were harvested 48 hours later and total RNA was purified by the RNeasy™ kit (Qiagen, Valencia, Calif.). cDNA was produced using ProtoScript First Strand cDNA Synthesis Kit (New England Biolabs, Inc., Beverly, Mass.). mRNA quantitation was performed by real-time PCR, using a iCycler™ (Bio-Rad Laboratories, Hercules, Calif.) and TaqMan Gene Expression Assay™ kits (Applied Biosystems, Foster City, Calif.) for Erk-2, 1 and reference genes: Akt-1 and transferrin receptor (TFRC) by which the relative expression values were calculated using the delta Ct method (Pfaffl Nucl. Acids Res. 29(9):e45 (2001)). Error bars in FIG. 9(c) represent standard deviation of three independent experiments.

Analysis of the two transcript profiles revealed gene-specific targeting of Erk-2 mRNA with Erk-2 hsiRNA mix with no effect on the Erk1 mRNA levels even at high concentrations of transfected hsiRNA. Conversely, high amounts of the Erk1 hsiRNA mix did not affect Erk2 mRNA levels. These results showed that hsiRNA mixtures can be highly potent (FIG. 9C) and highly specific (FIG. 9D).

1. A composition, comprising:
a plurality of double-stranded RNA (dsRNA) fragments having overlapping sequences, each fragment having a size in the range of 18-30 nt, wherein the composition is formed by enzymatic digestion of one or more large dsRNAs and wherein less than 2 nM of the composition is capable of specifically silencing expression of a target gene by at least 65% in transfected COS cells.

2. A composition according to claim 1, wherein the composition is capable of specifically silencing target gene expression by 70%.

3. A composition according to claim 1, wherein the composition is capable of specifically silencing target gene expression by 80%.

4. A composition according to claim 1, wherein the dsRNA has a size of at least 100 nt in length.

5. A composition according to claim 1, wherein the plurality of fragments is at least 5 fragments.

6. A composition according to claim 1, wherein the plurality of fragments is at least 10 fragments.

7. A composition according to claim 1, wherein the large dsRNA has a sequence identity with a first portion of a messenger RNA (mRNA) sequence such that the plurality of dsRNA fragments derived therefrom has a greater gene silencing activity at less than 2 nM than a second plurality of fragments having sequence identity with a second portion of the mRNA.

8. A composition according to claim 1, wherein the plurality of dsRNA fragments have a greater gene silencing activity at a concentration of less than 2 nM than any single fragment in the composition.

9. A composition according to claim 1, wherein the enzymatic digestion is achieved using RNaseIII in a manganese buffer or a mutant RNaseIII.

10. A composition according to claim 1, wherein the target gene encodes Erk1 or Erk2 and the large dsRNA has sequence identity with a portion of mRNA transcribed from the gene.

11. A composition according to claim 1, wherein the target gene encodes Ffluc or Renilla luciferase and the large dsRNA has sequence identity with a portion of mRNA transcribed from the gene.

12. A composition according to claim 1, wherein the fragments are derived from digestion of a plurality of dsRNAs and wherein the plurality of dsRNA have sequence identity with non-contiguous regions of the mRNA.

13. A composition according to claim 1, wherein the fragments are derived from digestion of a plurality of dsRNA wherein the plurality of dsRNA has sequence identity with contiguous regions of the mRNA.

14. A composition according to claim 1, wherein 1 nM of the composition is capable of silencing gene expression by at least 70%.

15. A method of preparing a composition described in claim 1, comprising:
(a) transcribing at least one RNA molecule having a sequence identity with a portion of a target gene, to form a large dsRNA;
(b) cleaving the large dsRNA into a mixture of overlapping fragments having a size in the range of 18-30 nt by means of RNaseIII or mutants thereof;
(c) determining whether less than 2 nM of the large dsRNA can silence at 65% of gene expression of the target gene in COS cells after transfection; and
(d) obtaining the composition described in claim 1.

16. A method of silencing gene expression; comprising:
(a) cleaving with an enzyme, a large dsRNA having sequence identity with a target gene, wherein the enzyme is RNaseIII or a mutant thereof and the cleavage product is a set of overlapping fragments of dsRNA in which greater than 80% of the fragments have a size of less than about 40 nt;
(b) transfecting cells with the cleavage product of step (a) without size fractionating the product; and
(c) obtaining at least 65% silencing of expression of the target gene.

17. A method according to claim 16, wherein step (b) further comprises: transfecting cells with less than 2 nM of the cleavage product of step (a).

18. A method according to claim 16, wherein RNase III cleavage is achieved in the presence of manganese buffer.
